(19) United States
(12) Patent Application Publication
(10) Pub. No.: US 2008/0228016 A1
(43) Pub. Date: Sep. 18, 2008

(54) Method for Chlorinating Alcohols

(75) Inventors: Thorsten Rohde, Mannheim (DE); Oliver Huttenloch, Ispringen (DE); Friederike Osswald, Mannheim (DE); Kathrin Wissel, Bensheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE

(21) Appl. No.: 12/066,321

(22) PCT Filed: Aug. 31, 2006
(86) PCT No.: PCT/EP2006/065873

§ 371 Date: Mar. 10, 2008

(30) Foreign Application Priority Data
Sep. 10, 2005 (DE) 10 2005 043 141.0

(51) Int. Cl. C07C 17/16

(52) U.S. Cl. 570/261

(57) ABSTRACT

A process for preparing organic chlorides in which the chlorine atom is bonded to a CH₂ group by reacting the corresponding alcohols with thionyl chloride in the presence of a triaylphosphine oxide at a temperature of from 20 to 200° C. and a pressure of from 0.01 to 10 MPa abs, which comprises using the triarylphosphine oxide in a molar ratio to the amount of OH groups to be chlorinated of from 0.0001 to 0.5.

The present invention relates to a process for preparing organic chlorides in which the chlorine atom is bonded to a CH₂ group by reacting the corresponding alcohols with thionyl chloride in the presence of a triarylphosphine oxide at a temperature of from 20 to 200° C. and a pressure of from 0.01 to 10 MPa abs.

Alkyl chlorides are important intermediates in the synthesis of chemical products, for example of dyes, active pharmaceutical and agricultural ingredients, electroplating assistants, ligands for homogeneous catalysts, disinfectants, steroids and growth hormones.

The chlorinating of alcohols with chlorinating agents, for example thionyl chloride, phosphorus trichloride or phosgene is common knowledge. Preference is given to using chlorination catalysts for this purpose.

U.S. Pat. No. 2,331,681 describes the chlorinating of glycolonitrile to chloroacetonitrile with thionyl chloride in the presence of the organic bases pyridine, dimethylaniline and quinoline. It is unfavorable that equimolar amounts of base are required for this purpose and that they have to be removed and disposed of after the reaction.

EP-A 0 645 357 teaches a process for preparing alkyl chlorides from the corresponding alcohol and a stoichiometric amount of a catalyst adduct (“Vilsmeier salt”) formed from an N,N-dialkylformamide and phosgene or thionyl chloride. Disadvantages of this process are the use of equimolar amounts of catalyst and the gradual feeding of alcohol and of the chlorinating agent.

GB-A 2,182,039 discloses the chlorination of alcohols with thionyl chloride or phosgene in the presence of triphenylphosphine oxide or triphenylphosphine sulfide. Examples VIII and IX describe the chlorination of 2,3,6,3′,4′-pentaacetylsucrose with thionyl chloride in the presence of triphenylphosphine oxide and toluene or 1,2-dichloroethane. A molar ratio of triphenylphosphine oxide used to the amount of OH groups to be chlorinated of about 0.7 is calculated from example VIII and of about 1.7 from example IX.

DE-A 41 16 365 teaches the preparation of alkyl, alkenyl and alkynyl chlorides by reacting the corresponding alcohols with phosgene or thionyl chloride in the presence of an aliphatic, cycloaliphatic or cyclic-aliphatic phosphine oxide as a catalyst. It is emphasized on page 2, lines 10 to 12 that, according to the above-cited GB-A 2,182,039 document, triarylphosphine oxides have to be used in superstoichiometric amounts owing to their low reactivity and, owing to these high amounts and the low solubility, complicate the workup of the reaction mixture. However, a disadvantage of the use of aliphatic, cycloaliphatic and cyclic-aliphatic phosphine oxides is their poorer availability, especially compared to triphenylphosphine oxide, owing to complicated preparation processes, which is also reflected in the price from an economic point of view.

It was an object of the present invention to find a process for preparing organic chlorides in which the chlorine atom is bonded to a CH₂ group, which does not have the disadvantages of the prior art, requires easily obtainable and industrially readily available reactants and, if appropriate, only easily obtainable and industrially readily available assistants/catalysts, leads to a high conversion, a high selectivity and a high space-time yield of product of value under mild temperatures and pressures, enables simple workup of the reaction mixture, and in which the product of value can be obtained in high purity.

Accordingly, a process has been found for preparing organic chlorides in which the chlorine atom is bonded to a CH₂ group by reacting the corresponding alcohols with thionyl chloride in the presence of a triarylphosphine oxide at a temperature of from 20 to 200° C. and a pressure of from 0.01 to 10 MPa abs, which comprises using the triarylphosphine oxide in a molar ratio to the amount of OH groups to be chlorinated of from 0.0001 to 0.5.

On the basis of the technical teachings of DE-A 41 16 365 and GB-A 2,182,039, according to which triarylphosphine oxides have to be used in superstoichiometric amounts owing to their low reactivity, it was entirely surprising that, for the preparation of organic chlorides in which the chlorine atom is bonded to a CH₂ group by reacting the corresponding alcohols with thionyl chloride, specifically substoichiometric amounts of triarylphosphine oxide are absolutely sufficient.

In the process according to the invention, the triarylphosphine oxide is used preferably in a molar ratio to the amount of OH groups to be chlorinated of from 0.001 to 0.5, more preferably from 0.001 to 0.1, and most preferably from 0.005 to 0.05.

In the process according to the invention, the alcohol used is generally a compound of the general formula (I)

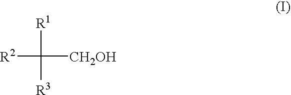

in which the R¹ to R³ radicals are each independently
hydrogen or a carbon-comprising organic radical which is saturated or unsaturated, acyclic or cyclic, aliphatic, aromatic or araliphatic, unsubstituted, or interrupted or substituted by from 1 to 5 heteroatoms or functional groups and has from 1 to 30 carbon atoms;
two radicals together are a divalent carbon-comprising organic radical which is saturated or unsaturated, acyclic or cyclic, aliphatic, aromatic or araliphatic, unsubstituted, or interrupted or substituted by from 1 to 5 heteroatoms or functional groups and has from 1 to 40 carbon atoms; or
all three radicals together are a trivalent carbon-comprising organic radical which is saturated or unsaturated, acyclic or cyclic, aliphatic, aromatic or araliphatic, unsubstituted, or interrupted or substituted by from 1 to 5 heteroatoms or functional groups and has from 1 to 50 carbon atoms.

Useful heteroatoms are in principle all heteroatoms which are capable in a formal sense of replacing a —CH₂—, a —CH═, a —C≡ or a ═C═ group. When the carbon-comprising radical comprises heteroatoms, preference is given to oxygen, nitrogen, sulfur, phosphorus and silicon. Preferred groups include especially —O—, —S—, —SO—, —SO₂—, —NR′—, —N═, —PR′—, —PR′₂ and —SiR′₂—, where R′ radicals are the remaining portion of the carbon-comprising radical.

Useful functional groups are in principle all functional groups which can be bonded to a carbon atom or a heteroatom. Suitable examples include —OH (hydroxyl), -Hal (halogen), ═O (especially as a carbonyl group), —NH₂ (amino), ═NH (imino), —COOH (carboxyl), —CONH₂ (carboxamide), —SO₃H (sulfo) and —CN (cyano). Functional groups and heteroatoms may also be directly adjacent, so that combinations, for instance —COO-(ester), —CONH— (secondary amide) or —CONR′— (tertiary amide), are also included. Halogens (Hal) include fluorine, chlorine, bromine and iodine.

The R¹ to R³ radicals are preferably each independently
hydrogen;
C₁- to C₂₀-alkyl which is optionally substituted by functional groups, cycloalkyl, aryl, aryloxy and/or alkyloxy and/or interrupted by one or more oxygen atoms;
C₅- to C₁₂ cycloalkyl optionally substituted by functional groups, cycloalkyl, aryl, aryloxy and/or alkyloxy;
C₂- to C₂₀-alkenyl which is optionally substituted by functional groups, cycloalkyl, aryl, aryloxy and/or alkyloxy and/or interrupted by one or more oxygen atoms, and may comprise one or more C═C double bonds;
C₅- to C₁₂-cycloalkenyl optionally substituted by functional groups, cycloalkyl, aryl, aryloxy and/or alkyloxy;
C₂- to C₂₀-alkynyl which is optionally substituted by functional groups, cycloalkyl, aryl, aryloxy and/or alkyloxy and/or interrupted by one or more oxygen atoms, and may comprise one or more C≡C triple bonds; or
C₆- to C₁₂-aryl optionally substituted by functional groups, cycloalkyl, aryl, alkyl, aryloxy and/or alkyloxy;
   or two radicals together are
C₄- to C₃₀-alkylene which is optionally substituted by functional groups, cycloalkyl, aryl, aryloxy and/or alkyloxy and/or interrupted by one or more oxygen atoms; or
C₅- to C₃₀-alkenylene which is optionally substituted by functional groups, cycloalkyl, aryl, aryloxy and/or alkyloxy and/or interrupted by one or more oxygen atoms, and may comprise one or more C═C double bonds;
   or all three radicals together are
a trivalent, saturated or unsaturated C₆- to C₄₀ hydrocarbon radical which is optionally substituted by functional groups, cycloalkyl, aryl, aryloxy and/or alkyloxy and/or interrupted by one or more oxygen atoms.

C₁- to C₂₀-alkyl which is optionally substituted by functional groups, cycloalkyl, aryl, aryloxy and/or alkyloxy and/or interrupted by one or more oxygen atoms is, for example, methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, 2-ethylhexyl, 2,4,4-trimethylpentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl, nonadecyl, eicosyl, cyclopentylmethyl, 2-cyclopentylethyl, 3-cyclopentylpropyl, cyclohexylmethyl, 2-cyclohexylethyl, 3-cyclohexylpropyl, benzyl (phenylmethyl), diphenylmethyl (benzhydryl), triphenylmethyl, 1-phenylethyl, 2-phenylethyl, 3-phenylpropyl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di-(methoxycarbonyl)ethyl, methoxy, ethoxy, 1,3-dioxolan-2-yl, 1,3-dioxan-2-yl, 2-methyl-1,3-dioxolan-2-yl, 4-methyl-1,3-dioxolan-2-yl, hydroxymethyl, 2-hydroxyethyl, 2-hydroxypropyl, 3-hydroxypropyl, 4-hydroxybutyl, 6-hydroxyhexyl, 2-hydroxymethyl-2-propyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl, 6-ethoxyhexyl, acetyl, chloromethyl, 2-chloroethyl, trichloromethyl, 1,1-dimethyl-2-chloroethyl, methoxymethyl, 2-butoxyethyl, diethoxymethyl, diethoxyethyl, 2-isopropoxyethyl, 2-butoxypropyl, 2-octyloxyethyl, 2-methoxyisopropyl, 2-(methoxycarbonyl)ethyl, 2-(ethoxycarbonyl)ethyl, 2-(n-butoxycarbonyl)ethyl, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-dioxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-dioxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 1′-ethoxy-4,8-dioxaundecyl, 15-ethoxy-4,8,12-trioxapentadecyl, 9-ethoxy-5-oxanonyl or 14-ethoxy-5,10-oxatetradecyl.

C₅- to C₁₂-cycloalkyl optionally substituted by functional groups, cycloalkyl, aryl, aryloxy and/or alkyloxy is, for example, cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, chlorocyclohexyl, dichlorocyclohexyl or dichlorocyclopentyl.

C₂- to C₂₀-alkenyl which is optionally substituted by functional groups, cycloalkyl, aryl, aryloxy and/or alkyloxy and/or interrupted by one or more oxygen atoms is, for example, vinyl, 2-propenyl, 3-butenyl, cis-2-butenyl or trans-2-butenyl.

C₅- to C₁₂-cycloalkenyl optionally substituted by functional groups, cycloalkyl, aryl, aryloxy and/or alkyloxy is, for example, 3-cyclopentenyl, 2-cyclohexenyl, 3-cyclohexenyl or 2,5-cyclohexadienyl.

C₂- to C₂₀-Alkynyl which is optionally substituted by functional groups, cycloalkyl, aryl, aryloxy and/or alkyloxy and/or interrupted by one or more oxygen atoms is, for example, ethynyl, 1-propynyl or 2-propynyl.

C₆- to C₁₂-aryl optionally substituted by functional groups, cycloalkyl, aryl, alkyl, aryloxy and/or alkyloxy is, for example, phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2-nitrophenyl, 4-nitrophenyl, 2,4-dinitrophenyl, 2,6-dinitrophenyl, 4-dimethylaminophenyl, methoxyethylphenyl or ethoxymethylphenyl.

When two adjacent radicals together form a C₄- to C₃₀-alkylene or C₅- to C₃₀-alkenylene radical which is optionally substituted by functional groups, cycloalkyl, aryl, aryloxy and/or alkyloxy and/or interrupted by one or more oxygen atoms, it is, for example, 1,3-propylene, 1,4-butylene, 1,5-pentylene, 2-oxa-1,3-propylene, 1-oxa-1,3-propylene, 2-oxa-1,3-propylene, 1-oxa-1,3-propenylene or 3-oxa-1,5-pentylene.

When all three radicals together form a trivalent, saturated or unsaturated C₆- to C₄₀-hydrocarbon radical which is optionally substituted by functional groups, cycloalkyl, aryl, aryloxy and/or alkyloxy and/or interrupted by one or more oxygen atoms, it is, for example, an unsubstituted or substituted radical of the general formula (II)

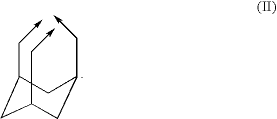

When the abovementioned radicals comprise heteroatoms, generally at least one carbon atom, preferably at least two carbon atoms, is/are disposed between two heteroatoms.

In the process according to the invention, the alcohols used are methanol, ethanol, 1-propanol, 1-butanol, 1-pentanol, 1-hexanol, 1-heptanol, 1-octanol, 1-nonanol, 1-decanol, propargyl alcohol, 2-butene-1,4-diol, 2-butyn-1,4-diol, 1,2-ethanediol, 1,2-propanediol, 1,3-propanediol, 1,2-butanediol, 1,4-butanediol, 1,5-pentanediol, 1,6-hexanediol, 1,2-pentanediol, 1,2,4-butanetriol, ethylene glycol monomethyl ether, ethylene glycol monoethyl ether, diethylene glycol, diethylene glycol monomethyl ether, diethylene glycol monoethyl ether, triethylene glycol, triethylene glycol monomethyl ether, triethylene glycol monoethyl ether, tetraethylene glycol, tetraethylene glycol monomethyl ether, tetraethylene glycol monoethyl ether, pentaethylene glycol, pentaethylene glycol monomethyl ether, pentaethylene glycol monoethyl ether, hexaethylene glycol, hexaethylene glycol monomethyl ether, hexaethylene glycol monoethyl ether, butylene glycol monomethyl ether, butylene glycol monoethyl ether, butylene glycol monobutyl ether, dibutylene glycol, dibutylene glycol monomethyl ether, dibutylene glycol monoethyl ether, dibutylene glycol monobutyl ether, tributylene glycol, tributylene glycol monomethyl ether, tributylene glycol monoethyl ether, tributylene glycol monobutyl ether, tetraethylene glycol, tetraethylene glycol monomethyl ether, tetraethylene glycol monoethyl ether, tetrabutylene glycol monobutyl ether, benzyl alcohol, 2-hydroxymethylbenzyl alcohol, 3-hydroxymethylbenzyl alcohol, 4-hydroxymethylbenzyl alcohol, 4-methoxybenzyl alcohol, 2,2-dimethylpropanol, 2-ethylhexanol, 2-ethylheptanol, 2-propylheptanol, 2-propylheptanol, pentaerythritol, 2,2-dimethyl-1,3-propanediol, 1,1,1-tris(hydroxymethyl)ethane or 1,1,1-tris(hydroxymethyl)propane.

More preferably, the R¹ to R³ radicals are each independently
hydrogen;
C₁- to C₂₀-alkyl which is optionally substituted by hydroxyl, halogen, cycloalkyl, aryl, aryloxy and/or alkyloxy and/or interrupted by one or more oxygen atoms;
C₂- to C₂₀-alkenyl which is optionally substituted by hydroxyl, halogen, cycloalkyl, aryl, aryloxy and/or alkyloxy and/or interrupted by one or more oxygen atoms, and may comprise one or more C═C double bonds;
C₂- to C₂₀-alkynyl which is optionally substituted by hydroxyl, halogen, cycloalkyl, aryl, aryloxy and/or alkyloxy and/or interrupted by one or more oxygen atoms, and may comprise one or more C≡C triple bonds; or
C₆- to C₁₂-aryl optionally substituted by hydroxyl, halogen, cycloalkyl, aryl, alkyl, aryloxy and/or alkyloxy.

The alcohol used is most preferably a compound of the general formula (I) in which not more than one and most preferably no radical from R¹ to R³ is hydrogen.

In particular, the R¹ to R³ radicals are each independently
C₁- to C₂₀-alkyl which is optionally substituted by hydroxyl and/or halogen and/or interrupted by one or more oxygen atoms; or
C₆- to C₁₂-aryl optionally substituted by a hydroxyl and/or halogen.

The alcohols used in the process according to the invention are most preferably 1,6-hexanediol, 2,2-dimethyl-1,3-propanediol, 1,1,1-tris(hydroxymethyl)ethane, 2-ethylhexanol, propargyl alcohol and especially 1,6-hexanediol, 2,2-dimethyl-1,3-propanediol and 1,1,1-tris(hydroxymethyl)ethane.

In the process according to the invention, the catalyst used may be either mono- or poly-ring-substituted triarylphosphine oxides or unsubstituted triphenylphosphine oxide. Preference is given to using triarylphosphine oxides of the general formula (III)

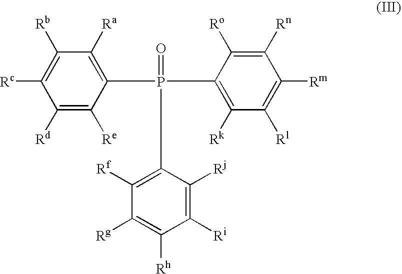

in which the R^{a} to R^{o} radicals are each independently hydrogen or C₁- to C₆-alkyl. Very particular preference is given to using triphenylphosphine oxide.

The alcohol, the thionyl chloride, the triarylphosphine oxide and any solvent to be used can be added in different ways and in a different sequence. The alcohol may, for example, be used in liquid form, in solid form or dissolved in an inert solvent. It is also possible to add a portion or the entire amount of triarylphosphine oxide dissolved in or mixed with the alcohol.

The thionyl chloride is generally added in liquid form. In addition, it is also possible in principle to dissolve a portion or the entire amount of triarylphosphine oxide in the thionyl chloride and to add it in this way.

Alternatively to the abovementioned addition methods for the triarylphosphine oxide, it is also possible to add it separately, if appropriate dissolved in an inert solvent.

In general, it is not necessary to add a solvent, especially not when the alcohol used and/or the reaction mixture formed by the reaction is/are present in liquid form at the selected reaction temperature. In the case of reaction mixtures which would be present in solid form at the selected reaction temperature, the use of an inert solvent is, however, generally advisable. An inert solvent is understood to mean solvents which are chemically inert toward the alcohol used, the thionyl chloride, the organic chloride formed and the triarylphosphine oxide used. “Chemically inert” means that the diluents do not react chemically with the substances mentioned under the conditions selected. Examples of suitable solvents include aromatic or aliphatic hydrocarbons. When a solvent is used, its boiling point is preferably sufficiently far removed from the boiling point of the desired organic chloride in order subsequently to be able to be remove the product of value in a simple manner and in the desired purity. The amount of solvent used is preferably in the range from 100 to 1000% of the amount which is required to keep the reaction mixture in the liquid phase.

In the process according to the invention, preference is given to initially charging the alcohol and the triarylphosphine oxide and bringing them to the desired reaction temperature. Subsequently, the introduction of thionyl chloride, which is typically added in liquid form, is generally commenced.

In the process according to the invention, the thionyl chloride is used generally in a molar ratio to the amount of OH groups to be chlorinated or from 0.9 to 5, preferably from 0.95 to 2, more preferably from 0.99 to 1.5 and most preferably from 1 to 1.2.

The hydrogen chloride and sulfur dioxide reaction gases released in the reaction are generally removed continuously from the reaction apparatus.

The process according to the invention is generally operated semicontinuously or continuously. In semicontinuous mode, the alcohol is typically initially charged in a suitable reaction apparatus and the entire amount of triarylphosphine oxide or at least a portion thereof is dissolved therein. Subsequently, the thionyl chloride which, if appropriate, comprises the remaining amount of the triarylphosphine oxide, is added continuously in accordance with the progress of the reaction.

In continuous mode, the reactants and the triarylphosphine oxide are typically fed simultaneously to a suitable reaction apparatus, and an amount corresponding to the amount fed is simultaneously removed from the reaction apparatus.

Suitable reaction apparatus includes in principle all reaction apparatus which is suitable for liquid/liquid reactions, for example stirred tanks.

The process according to the invention is carried out at a pressure of from 0.01 to 10 MPa abs, preferably from 0.05 to 5 MPa abs, more preferably from 0.09 to 0.5 MPa abs and most preferably from 0.09 to 0.2 MPa abs.

In addition, the process according to the invention is carried out at a temperature of from 20 to 200° C., preferably from 30 to 180° C. and more preferably from 50 to 160° C.

Once the desired amount of thionyl chloride has been added, the resulting reaction solution is generally left under the reaction conditions for postreaction for a certain time, generally from 30 minutes to 6 hours. In order to remove or deplete excess thionyl chloride and its hydrogen chloride and sulfur dioxide reaction products from the reaction solution, it is possible, if appropriate, to pass inert gas through while mixing (“stripping”).

The reaction effluent is generally worked up by the known methods. The desired organic chloride is preferably isolated by fractional distillation. If required, the triarylphosphine oxide used can be recovered by distillation and reused.

In a general embodiment for semicontinuously preparing organic chlorides in which the chlorine atom is bonded to a CH₂ group, the desired amount of the corresponding alcohol and the desired amount of triarylphosphine oxide are introduced into a stirred tank with reflux condenser and the mixture is brought to the desired reaction temperature. Subsequently, the addition of thionyl chloride is commenced, the rate of addition generally being adjusted such that the unconverted thionyl chloride boils gently under reflux. Once the addition of the desired amount of thionyl chloride has ended, the reaction mixture is left for postreaction with further stirring for from about 0.5 to 6 hours. Advantageously, residual hydrogen chloride and residual sulfur dioxide are subsequently stripped out with nitrogen. Finally, the reaction mixture is fed to a distillation column in which first the excess thionyl chloride and then, preferably under reduced pressure, the desired organic chloride are distilled off.

The process according to the invention enables the preparation of organic chlorides in which the chlorine atom is bonded to a CH₂ group, the reactants to be used being alcohols which are generally easily obtainable and industrially readily available, and also easily obtainable and industrially readily available thionyl chloride, and the catalyst to be used is preferably very readily available triphenylphosphine oxide. In addition, the process found also leads under mild temperatures and pressures to a high conversion, a high selectivity and a high space-time yield of product of value, and enables simple workup of the reaction mixture, the product of value being obtainable in high purity.

### EXAMPLES

### Example 1

### Comparative Example without Catalyst

A 250 ml four-neck stirred apparatus with condenser, bubble counter, dropping funnel, thermometer and blade stirrer was initially charged with 31.20 g (0.30 mol) of 2,2-dimethyl-1,3-propanediol (neopentyl glycol) at room temperature and heated to 80° C. in an oil bath. Thionyl chloride was then added dropwise with stirring to the solid reactant. The reaction commenced immediately, which was shown by the vigorous evolution of gas. In the course of this, the internal temperature rose to 85° C. and the solid 2,2-dimethyl-1,3-propanediol began to be converted to a liquid. After addition of about 44.6 g (0.375 mol) of thionyl chloride within one hour, in spite of further dropwise addition of thionyl chloride, no further evolution of gas was observed. The internal temperature was then raised to 100° C. and further thionyl chloride was added dropwise up to a total amount of 89.25 g (0.75 mol) and a total dropwise addition time of about 2 hours, in the course of which still no further evolution of gas took place. The mixture was then stirred at an oil bath temperature of 130° C. for 3 hours, in the course of which the mixture refluxed at an internal temperature of about 111° C. Subsequently, the mixture was cooled to room temperature and analyzed by gas chromatography. It was not possible to detect any 1,3-dichloro-2,2-dimethylpropane, but rather mainly the cyclic ester formed from the diol and thionyl chloride.

### Example 2

### Inventive with Catalyst

An apparatus as described in example 1 was initially charged with 83.20 g (0.80 mol) of 2,2-dimethyl-1,3-propanediol (neopentyl glycol) and 4.73 g (0.017 mol) of triphenylphosphine oxide at room temperature and heated to 80° C. in an oil bath (molar ratio of triphenylphosphine oxide to the amount of OH groups to be chlorinated equals 0.011). In the course of this the two substances melted slowly to become intermixed with streak formation. Thionyl chloride was then added dropwise with stirring to the opaque melt. The reaction commenced immediately, which was shown by the vigorous evolution of gas. Even after the first few drops of thionyl chloride, the reaction mixture was completely fluid. The internal temperature rose briefly to 86° C. Within about 2.5 hours, 100.57 g (0.85 mol) of thionyl chloride were added dropwise, and no further gas evolution was observed toward the end of this time span. The internal temperature was then raised to 100° C. without further dropwise addition of thionyl chloride, and gentle evolution of gas was observable from about 95° C. At about 100° C., further thionyl chloride was then added dropwise up to a total amount of 238.00 g (2.00 mol) and a total dropwise addition time of about 4.5 hours. The mixture was stirred further at an oil bath temperature of 130° C. and an internal temperature of about 115° C. for 2.5 hours, in the course of which gas evolution was still observable. Toward the end of the continued stirring time, however, the evolution of gas abated gradually. Subsequently, the mixture was cooled to room temperature. 141.41 g of reaction effluent were obtained. This was analyzed by gas chromatography and the main product detected was 1,3-dichloro-2,2-dimethylpropane. The reaction effluent was then fractionally distilled under a reduced pressure of 13 hPa abs (13 mbar abs). The result is reproduced in table 1. The three fractions gave rise to a total mass of dichloro-2,2-dimethylpropane of 112.79 g, which corresponds to a yield of 99.89%.

TABLE 1

| | Fraction 1 | Fraction 2 | Fraction 3 |
|---|---|---|---|
| Distillation temperature [° C.] | 55 | 55 | 55 |
| Bottom temperature [° C.] | 75 | 75 | 75 |
| Mass of the fraction [g] | 48.70 | 42.02 | 24.07 |
| Purity [GC area %] | 97.2 | 99.5 | 98.3 |
| Mass of 1,3-dichloro-2,2- | 47.33 | 41.80 | 23.66 |
| dimethylpropane [g] | | | |

### Example 3

### Inventive with Catalyst

An apparatus as described in example 1 was initially charged with 50.51 g (0.416 mol) of 1,1,1-tris(hydroxymethyl)ethane and 3.820 g (0.014 mol) of triphenylphosphine oxide at room temperature and heated to 135° C. in an oil bath (molar ratio of triphenylphosphine oxide to the amount of OH groups to be chlorinated equals 0.011). In the course of this, the two substances melted to become intermixed with one another with streak formation. 10 ml of thionyl chloride were then added dropwise to the opaque melt with stirring. The reaction commenced immediately, which was shown by the vigorous evolution of gas. Even after the first few drops of thionyl chloride, the reaction mixture was completely fluid. Within about 20 min, 101.5 ml (2.838 mol) of thionyl chloride were added dropwise at from 110 to 120° C. The mixture was stirred at a temperature of 111° C. for a further 90 min. Subsequently, the mixture was cooled to room temperature. 83.89 g of reaction effluent were obtained as a clear light brown liquid. This was analyzed by gas chromatography and the main product detected was 1,3-dichloro-2-chloromethyl-2-methylpropane. The reaction effluent was then fractionally distilled at a reduced pressure of 13 hPa abs (13 mbar abs). The result is reproduced in table 2. The product was obtained in a purity of 98% and a yield of 85%.

TABLE 2

| | Fraction |
|---|---|
| Distillation temperature [° C.] | 74 |
| Bottom temperature [° C.] | 76 |
| Mass of the fraction [g] | 63.2 |
| Purity [GC area %] | 98 |
| Mass of 1,3-dichloro-2,2-dimethylpropane [g] | 61.9 |

Examples 2 and 3 show that it is also possible by the process according to the invention to obtain 1,3-dichloro-2,2-dimethylpropane and 1,3-dichloro-2-chloromethyl-2-methylpropane, as sterically hindered di- or trichlorides, in very high yield and purity.

### Example 4

### Inventive with Catalyst

An apparatus as described in example 1 was initially charged with 51.54 g (0.423 mol) of 1,6-hexanediol and 4.101 g (0.014 mol) of triphenylphosphine oxide at room temperature and heated to 45° C. in an oil bath (molar ratio of triphenylphosphine oxide to the amount of OH groups to be chlorinated equals 0.017). In the course of this, both substances melted slowly to become intermixed with streak formation. Within about 95 min, 122.4 g (1.029 mol) of thionyl chloride were added dropwise at from 70 to 80° C. The mixture was stirred at a temperature of from 60 to 70° C. for another 85 min. Subsequently, the mixture was cooled to room temperature. A clear light brown liquid was obtained. This was analyzed by gas chromatography and the main product detected was 1,6-dichlorohexane. The reaction effluent was then fractionally distilled at a reduced pressure of 13 hPa abs (13 mbar abs). The result is reproduced in table 3. The product was obtained in a purity of >99% and a yield of 87%.

TABLE 3

| | Fraction |
|---|---|
| Distillation temperature [° C.] | 68 |
| Bottom temperature [° C.] | 69 |
| Mass of the fraction [g] | 57.5 |
| Purity [GC area %] | 98 |
| Mass of 1,3-dichloro-2,2-dimethylpropane [g] | 56.9 |

### Example 5

### Comparative Example with Phosgene as the Chlorinating Agent

An apparatus as described in example 1 was initially charged with 50 ml of 1,6-dichlorohexane together with 2 g (0.0072 mol) of triphenylphosphine oxide and heated to 115° C. Within 75 min, 80 g (1.24 mol) of gaseous phosgene and a suspension of 24 g (0.200 mol) of 1,1,1-tris(hydroxymethyl)ethane in 150 ml of 1,6-dichlorohexane were metered in (molar ratio of triphenylphosphine oxide to the amount of OH groups to be chlorinated equals 0.012). Subsequently, the mixture was stirred at 120° C. for a further hour. After the unconverted phosgene had been removed by introducing nitrogen, the resulting product was analyzed by gas chromatography. The reaction effluent comprised 2.5 GC area % of 1,3-dichloro-2-chloromethyl-2-methylpropane, 80 GC area % of mixtures of chloride-substituted chloroformates and 17.5 GC area % of cyclic carbonate.

Comparative example 5 shows that, in contrast to inventive example 3, small amounts of triarylphosphine oxide are insufficient in the chlorination of sterically hindered alcohols with phosgene and thus that the result with phosgene differs greatly from that with thionyl chloride.

1: A process for preparing organic chlorides in which the chlorine atom is bonded to a CH₂ group by reacting the corresponding alcohols with thionyl chloride in the presence of a triarylphosphine oxide at a temperature of from 20 to 200° C. and a pressure of from 0.01 to 10 MPa abs, which comprises using the triarylphosphine oxide in a molar ratio to the amount of OH groups to be chlorinated of from 0.0001 to 0.5.

2: The process according to claim 1, wherein the triarylphosphine oxide is used in a molar ratio to the amount of OH groups to be chlorinated of from 0.001 to 0.1.

3: The process according to claim 1, wherein the alcohol used is a compound of the general formula (I) 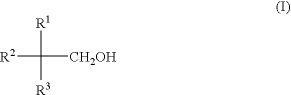
in which the R¹ to R³ radicals are each independently
hydrogen or a carbon-comprising organic radical which is saturated or unsaturated, acyclic or cyclic, aliphatic, aromatic or araliphatic, unsubstituted, or interrupted or substituted by from 1 to 5 heteroatoms or functional groups and has from 1 to 30 carbon atoms;
two radicals together are a divalent carbon-comprising organic radical which is saturated or unsaturated, acyclic or cyclic, aliphatic, aromatic or araliphatic, unsubstituted, or interrupted or substituted by from 1 to 5
heteroatoms or functional groups and has from 1 to 40 carbon atoms; or
all three radicals together are a trivalent carbon-comprising organic radical which is saturated or unsaturated, acyclic or cyclic, aliphatic, aromatic or araliphatic, unsubstituted, or interrupted or substituted by from 1 to 5 heteroatoms or functional groups and has from 1 to 50 carbon atoms.

4: The process according to claim 3, wherein the alcohol used is a compound of the general formula (I) in which the R¹ to R³ radicals are each independently
hydrogen;
C₁- to C₂₀-alkyl which is optionally substituted by hydroxyl, halogen,
cycloalkyl, aryl, aryloxy and/or alkyloxy and/or interrupted by one or more oxygen atoms;
C₂- to C₂₀-alkenyl which is optionally substituted by hydroxyl, halogen, cycloalkyl, aryl, aryloxy and/or alkyloxy and/or interrupted by one or more oxygen atoms, and may comprise one or more C═C double bonds;
C₂- to C₂₀-alkynyl which is optionally substituted by hydroxyl, halogen, cycloalkyl, aryl, aryloxy and/or alkyloxy and/or interrupted by one or more oxygen atoms, and may comprise one or more C≡C triple bonds; or
C₆- to C₁₂-aryl optionally substituted by hydroxyl, halogen, cycloalkyl, aryl, alkyl, aryloxy and/or alkyloxy.

5: The process according to claim 4, wherein the alcohol used is a compound of the general formula (I) in which not more than one radical from R¹ to R³ is hydrogen.

6: The process according to claim 5, wherein the alcohol used is a compound of the general formula (I) in which no radical from R¹ to R³ is hydrogen.

7: The process according to claim 6, wherein the alcohol used is 2,2-dimethyl-1,3-propanediol.

8: The process according to claim 1, wherein the triarylphosphine oxide used is triphenylphosphine oxide.

9: The process according to claim 1, wherein the thionyl chloride is used in a molar ratio to the amount of OH groups to be chlorinated of from 0.9 to 5.
